(19) United States
(12) Patent Application Publication
(10) Pub. No.: US 2005/0118229 A1
(43) Pub. Date: Jun. 2, 2005

(54) Implantable drug delivery device for sustained release of therapeutic agent

(75) Inventor: Anthony A. Boiarski, Hilliard, OH (US)

Correspondence Address: CALFEE HALTER & GRISWOLD, LLP, 800 SUPERIOR AVENUE, SUITE 1400, CLEVELAND, OH, 44114, US

(73) Assignee: iMEDD, Inc., Columbus, OH (US)

(21) Appl. No.: 10/969,679

(22) Filed: Oct. 20, 2004

Related U.S. Application Data
(60) Provisional application No. 60/512,969, filed on Oct. 21, 2003.

(51) Int. Cl. A61K 9/14

(52) U.S. Cl. 424/424

(57) ABSTRACT

An implantable drug-delivery device that utilizes a nanopore diffusion membrane in combination with a microporous hydration membrane for achieving long-term, zero-order release of a therapeutic compound or agent. The device comprises a housing, wherein the housing further comprises an interior chamber and at least one aperture passing through the housing; a nanopore membrane in communication with the housing and covering the aperture, wherein the nanopore membrane further comprises a plurality of nanopore channels formed therein and passing though the membrane; a microporous membrane disposed within the housing, the two membranes defining an interface therebetween; a first solvent, e.g., an aqueous medium, disposed within the interface and in communication with both the nanopore membrane and the microporous membrane; and a particulate composition contained within the microporous membrane, wherein the particulates are suspended or buoyant in a second solvent, e.g., a water-immiscible fluid, and wherein the second solvent is in communication with the microporous membrane.

### CROSS-REFERENCE TO RELATED APPLICATIONS

This patent application claims the benefit of U.S. Provisional Patent Application Ser. No. 60/512,969 filed on Oct. 21, 2003 entitled “Implantable Drug-Delivery Device Using a Particle-Hydration Membrane for Long-Term, Zero-Order Release” the disclosure of which is incorporated as if fully rewritten herein.

### STATEMENT REGARDING FEDERALLY FUNDED RESEARCH

This invention was not made by an agency of the United States Government nor under contract with an agency of the United States Government.

### TECHNICAL FIELD OF THE INVENTION

In general, this invention relates to devices and methods for drug delivery, and more specifically to an implantable drug-delivery device for delivery of a therapeutic agent over a predefined period of time.

### BACKGROUND OF THE INVENTION

For certain drugs that are effective at low dosages, e.g., therapeutic peptides, a desirable mode of delivery includes releasing the drug or other therapeutic agent from an implanted device over a sustained period of up to several months. In general, achieving effective, long-term drug-delivery utilizing an implantable device involves two primary challenges.

First, the amount of drug delivered by the implanted device should be substantially constant over time, thereby allowing the release profile to be close to zero order kinetics. Achieving close to zero order kinetics allows a treated individual to receive a substantially constant therapeutic dose over a predefined period of time without dose spiking or periods of sub-therapeutic delivery. The second challenge, particularly for therapeutic compounds that exhibit limited stability in an aqueous solution, is to contain the compound in a substantially stable form within the implantable device for periods up to six months prior to release. The reactivity of many drugs begins to decrease within about one week if the drugs are dissolved or suspended in an aqueous medium including, for example, the physiological medium of an implantation site. Thus, there is a need for an implantable device that may be utilized for extended-term delivery of a therapeutic agent that exhibits limited stability when dissolved in an aqueous medium or other solvent.

### SUMMARY OF THE INVENTION

The present invention provides an implantable drug-delivery device that utilizes a nanopore diffusion membrane in combination with a microporous hydration membrane for achieving long-term, zero-order release of a therapeutic compound or agent.

In a first aspect of the present invention, an implantable device for sustained delivery of a therapeutic agent comprises a housing, wherein the housing further comprises an interior chamber and at least one aperture passing through the housing; a nanopore membrane in communication with the housing and covering the aperture, wherein the nanopore membrane further comprises a plurality of nanopore channels formed therein and passing though the membrane; a microporous membrane disposed within the housing beneath the nanopore membrane, the two membranes defining an interface therebetween; a first solvent, e.g., an aqueous medium, disposed within the interface and in communication with both the nanopore membrane and the microporous membrane; and a particulate composition contained within the interior chamber below the microporous membrane, wherein the particulates are suspended in a mobile state in a second solvent, e.g., a water-immiscible fluid, and wherein the second solvent is in communication with the microporous membrane.

In a second aspect of the present invention, an implantable device for sustained delivery of a therapeutic agent comprises a housing, wherein the housing further comprises an interior chamber and at least one aperture passing through the housing; a nanopore membrane in communication with the housing and covering the aperture, wherein the nanopore membrane further comprises a plurality of nanopore channels formed therein and passing though the membrane; a microporous membrane disposed within the housing, wherein the microporous membrane further comprises a capsule, and wherein the two membranes define an interface therebetween; a first solvent (aqueous medium) disposed within the interface and in communication with both the nanopore membrane and the microporous membrane; and a particulate composition contained within the microporous membrane, wherein the particulates are buoyant within the second solvent (water-immiscible fluid), and wherein the second solvent is in communication with the microporous membrane.

Additional features and aspects of the present invention will become apparent to those of ordinary skill in the art upon reading and understanding the following detailed description of the exemplary embodiments.

### BRIEF DESCRIPTION OF THE DRAWINGS

The accompanying drawings, which are incorporated into and form a part of the specification, schematically illustrate one or more exemplary embodiments of the invention and, together with the general description given above and detailed description of the preferred embodiments given below, serve to explain the principles of the invention.

FIG. 1 is a cross-sectional view of a first exemplary embodiment of the drug-delivery device of the present invention.

FIG. 2 is a cross-sectional view of a second exemplary embodiment of the drug-delivery device of the present invention.

FIG. 3 is a graphical presentation of hydration data for the device of FIG. 1, wherein particle mass loaded in the device is a fixed quantity.

FIG. 4 is graphical presentation of hydration data for the device of FIG. 1, wherein the particle mass loaded in the device is decreased to both 25 percent and 10 percent of the mass used to obtain the data presented in FIG. 3.

FIG. 5 is a graphical presentation of hydration rate results in micrograms per day as a function of loaded particle mass for the device of FIG. 1.

FIG. 6 is a graphical presentation of hydration data for the device of FIG. 2, wherein particle mass loaded in the device is a fixed quantity.

### DETAILED DESCRIPTION OF THE INVENTION

In the context of the present invention, the term “therapeutic agent” refers to a biological or chemical agent used in the treatment of a disease or disorder. The phrase “agent stability in dehydrated form” refers to acceptable percentage of the agent's original biological activity (e.g., 80%) being retained for a period of at least three months at 37° C. when the agent is in a form where no water is present. A compound has “limited stability in aqueous form” if it loses more than 25% of its biological activity when stored in aqueous solution at 37° C. for 3 months. Typically a compound with limited stability in aqueous solution will lose more than about 50% of its activity under these storage conditions.

The term “nanopore channels” refers to a channel in which at least cross-sectional dimension is in the range of 4 to 50 nanometers. The other cross-sectional dimension is typically in the 2 to 50 micrometer range. The length dimension of the channels is typically in the 50 micrometer to 5 mm range. The term “substantially zero-order kinetics” refers to the principle that over an acceptable percentage of the dose of therapeutic agent loaded into an implantable device, the rate of release of the agent is approximately constant. The term “microporous hydration membrane” refers to a membrane has pores that are in the micrometer range (e.g., greater than 1 micrometer). Generally, the purpose of this membrane is to control the hyrdration rate of solid particles on one side of the membrane by limiting the surface area of the interface between a water-miscible liquid and an aqueous liquid.

The term “phase separation membrane” refers to a membrane that has been treated to render it hydrophobic; therefore, it can separate an aqueous (hydrophilic) medium from water-immiscible (hydrophobic) medium by retaining the aqueous phase while passing the water-immiscible phase through the membrane pores. The term “colloid” refers to a substance consisting of particles dispersed in another substance (e.g., liquid) where the particles are too small for resolution with an ordinary light microscope. The particles tend to stay in suspension for long periods of time because the settling velocity is typically very low. Finally, in this disclosure, the terms “particle” and “particulate” are used interchangeably.

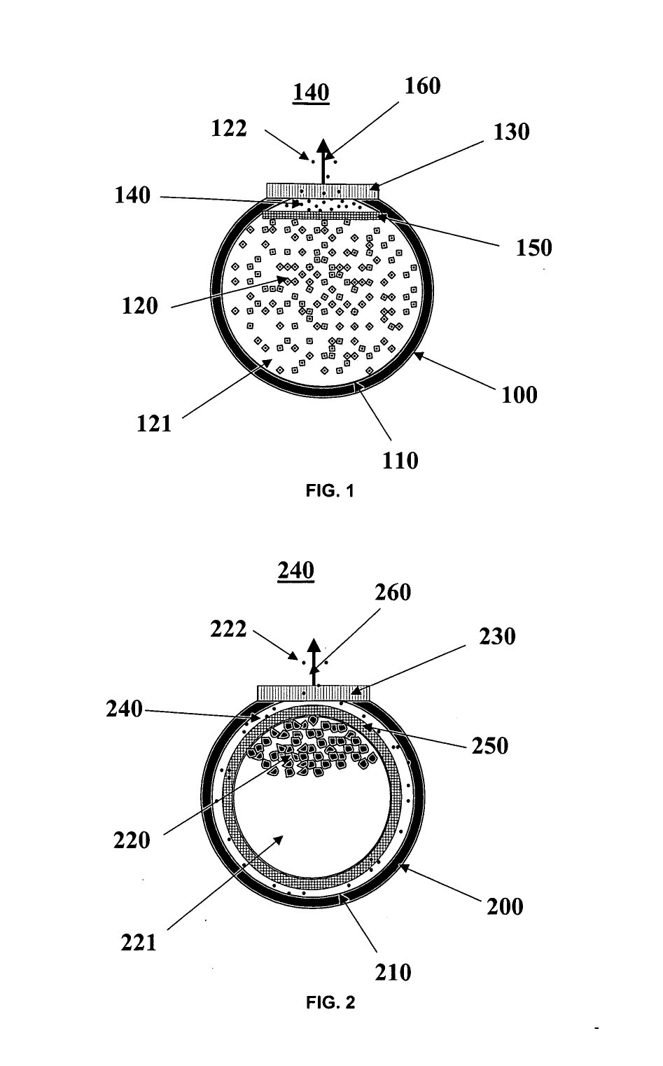

With reference to the Figures, FIG. 1 shows a first exemplary general embodiment of an implantable device for extended-term delivery of a therapeutic agent that has limited stability in a dissolved state. In this embodiment, the device includes a housing 100 that has an interior chamber 110 and a nanopore membrane 130. Nanopore memrane 130 includes a plurality of nanopore channels that are formed in and pass through the membrane and allow a first solvent, typically an aqueous medium 140, to flow into and out of the device. The nanopore channels typically have at least one cross-sectional dimension in the range between 4 and 50 nm and the general purpose of these nanopore channels is to control the diffusion of therapeutic agent in the dissolved state within an aqueous medium. The construction of this device provides diffusion control wherein the therapeutic agent is released from the implant with substantially zero-order kinetics, i.e., the release rate 160 is approximately constant during the time that a substantial percentage of the therapeutic agent loaded in chamber 110 is released.

As shown in FIG. 1, interior chamber 110 includes a reservoir that contains dehydrated particles 120 of the therapeutic agent suspended in a water-immiscible liquid 121. The liquid 121 is selected such that the dehydrated agent is stable, in a suspended form, in the liquid for an extended period of time substantially equivalent to the intended treatment time. Between the nanopore membrane 130 and the water-immiscible liquid 121 is a substantially planar, second membrane 150 that includes micro-sized pores. At the surface or within the porous structure of this microporous membrane 150 there is an interface. On one side of the interface is aqueous media 140, which is contained in the nanopore channels and upper portion of the chamber, and on the other side is water-immiscible liquid 121. The microporous membrane 150 has a fixed porosity and a pore size that acts to limit the effective area of the interface between the aqueous media and the water-immiscible liquid. The dehydrated particles 120 are limited to the interface for interacting with the aqueous media 140 in the chamber. This interaction typically results in a portion of the dehydrated particles dissolving in the aqueous media to produce a fixed, or at least predictable, amount of dissolved therapeutic agent 122. This dissolved agent then exits the device by zero-order diffusion through the nanopore membrane 130 at a substantially constant rate 160. Thus, the planar microporous membrane 150 acts as a time permissive, or rate-limiting barrier because it controls the hydration, and therefore the release of the therapeutic agent from the water-immiscible liquid to the aqueous medium by controlling the dissolution rate of the agent in contact with the interface.

In this embodiment, the dissolution rate of the dehydrated agent can be adjusted to be compatible with the diffusion rate of the agent through the nanopore channels by modifying the properties of membrane 150 by, for example, changing porosity, pore size, and/or membrane area, and the properties of the particles 120 by, for example, changing particle size. Adjusting the dissolution (i.e., hydration) rate to be approximately equal to the nanopore membrane diffusion rate restricts the time the therapeutic agent spends dissolved in the aqueous medium 140. Shortening the time period between dissolution and diffusion out of the device increases the likelihood that the therapeutic agent will remain stable because the time the agent spends in the aqueous media is minimized. This is important because many therapeutic agents have a finite, limited stability when dissolved in an aqueous media.

Examples of therapeutic agents 120 that are active at low concentration and must be administered subcutaneously over long time periods, but that have that finite or limited stability in aqueous media include: Interferon-alpha (2b) for the treatment of Hepatitis C, Interferon-beta for the treatment of Multiple Sclerosis, Alpha Epotin for treatment of chronic anemia, and Granulocyte Colony Stimulating Factor (GCSF) for treatment of neutropenia associated with cancer chemotherapy.

Selection of water-immiscible solvent 121 is based on several basic criteria: (i) the dehydrated therapeutic agent 120 should be stable in the solvent for time periods of about 3 to 6 months; (ii) the solvent should have a density of about 1-2 grams/cm³, which is the approximate density of the dehydrated agent particles; (iii) the solvent viscosity should be less than approximately 100 centipoise; and (iv) the solvent should be inherently stable. The water-immiscible liquid (i.e., fluid) solvent may be, for example, a fluorocarbon liquid, such as perfluorodecalin; an oil, such as olive or mineral oil; or a hydrocarbon liquid, such as benzyl benzoate. Stability of the therapeutic agent in the second solvent is likely if very little water is dissolved in the solvent. One method for removing residual dissolved water is use commercially available molecular sieves placed inside the implanted device or suspended in the solvent. The sieves will remove water and sequester it so it does not interact with the dehydrated therapeutic agent.

The solvent density limits described above are suitable for this embodiment of the present invention because it is desirable that the therapeutic agent particles 120 be suspended in the solvent 121 so as to freely interact with the hydration membrane 150. If the particles were not neutrally buoyant, they would typically sink to the bottom or float to the top of the interior chamber 110 (see FIG. 1). In one embodiment, the particles float to the top and contact the membrane (e.g., density of perfluorodecalin=2.9 g/cc and density of particles=1.1-1.5 g/cc). This contacting of the buoyant-particles and the hydration membrane allows the device to function in the orientation shown in FIG. 1. “Neutrally buoyant” refers to particles that have a very low settling velocity, Vs. The settling velocity of a particle suspended in a solvent is known in the art to be governed by the following equation:
V_{S}=g(ρ_{P}−ρ_{S})D_{P}²/18 η, (1)
where, g is the acceleration of gravity, ρ_{P} is the particle density, ρ_{S} is the solvent density, D_{P} is the particle size, and η is the solvent viscosity. Selecting a solvent where ρ_{P}˜ρ_{S} would result is a very low settling velocity for small particle sizes. For ρ_{P}=ρ_{S}, the particles would be buoyant (V_{S}=0) even for larger particle sizes. For ρ_{P}<ρ_{S} (the case where the solvent is a dense liquid), the particles will float.

The viscosity limits described above are desirable because it is preferable that the neutrally buoyant particles be mobile within reservoir 110 so that they will interact with the microporous membrane 150. The diffusion coefficient, D, for particles in a suspension is also known in the art to be represented as follows:
D=kT/(πηD_{P}), (2)
where, k is Boltzman's constant, T is the temperature in degrees Kelvin, η is the viscosity of the solvent, and D_{P} is the particle size. Selecting a solvent with a viscosity less than 100 centipoise increases the likelihood that particles with diameters less than 0.5 microns will be mobile (i.e., will diffuse) within the reservoir.

Incorporating some means of mixing the suspension within interior chamber 110 is used to enhance particle mobility in some embodiments of the present invention. For example, including one or more small balls or spheres in the reservoir provides a beneficial mixing effect. Molecular sieves may be used for this purpose, thereby providing a dual function of mixing and removing water from the solvent. The use of mixing may relax the discussed constraints placed on viscosity; thus, more viscous solvents (e.g., viscosities greater than 100 centipoise) may be compatible with this invention. Higher viscosities may be beneficial in terms of particle suspension because increased viscosity will result in decreased particle settling velocity according to Equation 1 (above).

Equations 1 and 2 (above) provide that particle diameters of less than 0.5 micrometers are preferred for the embodiment shown in FIG. 1. Such particles will provide: (i) long-lasting suspensions (i.e., particles come in contact with the hydrating membrane because they do not settle to the bottom of the chamber); and (ii) mobile particles that effectively diffuse within the implantable device reservoir, thereby effectively contacting the hydrating membrane. Particles in this size range are considered colloidal and certain milling techniques, known to those skilled in the art, are used to provide these colloidal suspensions. Certain additives known by those skilled in the art as “peptizing agents” may also be used to keep the suspended particles from aggregating.

In addition to creating and maintaining a colloidal suspension, other known methods may be used for stable, mobile suspensions of particles in a solvent. For example, larger particles (e.g., >50 μm) can be combined with a material that changes the overall combined particle density to make the particles neutrally buoyant. Again, small diameter balls could be placed in reservoir 110, to help mix or stir the larger particles and improve particle mobility, i.e., improve the probability of particle/hydrating membrane interaction.

Typically, the microporous, hydrating membrane 150 is selected based on its ability to control the rate that dehydrated therapeutic agent dissolves at the interface between the aqueous media and the water-immiscible liquid. Membrane properties affecting this rate include surface area, porosity, thickness, and pore size, where the pore size is larger than the particle size. Also, the water wetting characteristics of membrane material is important because hydrophilic and hydrophobic membranes can exhibit different behavioral characteristics. Hydrophobic phase separation membranes (e.g., Whatman 1PS) are useful for some embodiments because the interface area is more likely to be found on the upper side of the microporous membrane (see, for example, FIG. 1). Hydrophilic phase separation membranes are useful for other embodiments of the present invention.

The exemplary embodiment of the implantable device shown in FIG. 1 may be constructed using standard manufacturing practices. The nanopore membrane 130 is fabricated using silicon-based micro-processing techniques known in the art (see, for example, U.S. Pat. Nos. 5,651,900 5,770,076 5,798,042, 5,985,164, and 5,938,923). The microporous membranes are obtained commercially and are attached to the nanopore membrane using an adhesive or other appropriate attachment means. In production, the nanopore membrane may be encapsulated in a polymer holder and standard bonding techniques known in the art, such as ultrasonic bonding, can be used. Housing 100 can be molded or machined depending on the selected material. To avoid premature release of the particulate composition, the implant may stored without fluid (i.e., dry) or with the water-miscible fluid 121 present at locations within the device where the aqueous media 140 would normally be present. Before implantation, the device may be “primed” by introducing aqueous media into the nanopore channels and the interior portion of the device chamber that is in contact with the microporous membrane, i.e., the interface.

A second exemplary general embodiment of the present invention is shown in FIG. 2. This embodiment is similar to the first exemplary general embodiment; however, the shape and characteristics of the microporous membrane differ from that of the first general embodiment. The device includes a housing 200 that has an interior chamber 210 and a nanopore membrane 230. Nanopore membrane 230 includes a plurality of nanopore channels that are formed in and pass through the membrane and allow a first solvent, typically an aqueous medium 240, to flow into and out of the device. The nanopore channels typically have at least one cross-sectional dimension in the range between 4 and 50 nm and the general purpose of these nanopore channels is to control the diffusion of therapeutic agent in the dissolved state within an aqueous medium.

This embodiment includes a three-dimensional, capsule-like microporous hydration membrane 250 that forms a continuous, hollow membrane enclosure or packet within the device. This membrane packet provides a reservoir that contains dehydrated agent particles 220 suspended in a water-immiscible liquid 221. As with the first exemplary embodiment, the liquid selected as liquid 221 allows the dehydrated agent to be stable, in a suspended form, in the liquid for an extended period of time substantially equivalent to the intended treatment time.

The microporous membrane 250 is located between the nanopore membrane and the water-immiscible liquid 221. At the surface or within the porous structure of this microporous membrane 250 an interface is defined. On one side of the interface aqueous mediium 240 is contained in the nanopore channels and on the walls of the entire chamber, and on the other side of the interface is the water-immiscible liquid 221. This microporous membrane also has a fixed porosity and a pore size that acts to limit the effective area of the interface between the aqueous medium and the water-immiscible liquid. The interface allows the dehydrated particles 220 to interact with the aqueous medium in the chamber, and this interaction results in a portion of the dehydrated particle dissolving into the aqueous medium to produce a fixed amount of dissolved therapeutic agent 222. The dissolved agent then exits the device by substantially zero-order diffusion through the nanopore membrane 230 at a substantially constant rate 260. The cylindrical microporous membrane packet 250 thereby acts as a time-permissive barrier because it controls the hydration, and thus the release, of therapeutic agent from the water-immiscible liquid to the aqueous medium by controlling the dissolution rate of the agent in contact with the interface.

In this embodiment, the dissolution rate of the dehydrated agent can be adjusted to be compatible with the diffusion rate of the agent through the nanopore channels by modifying the properties of membrane 250 by, for example, changing porosity, pore size, and/or membrane area, and the properties of the particles 220 by, for example, changing particle size. Adjusting the dissolution (i.e., hydration) rate to be approximately equal to the nanopore membrane diffusion rate restricts the time the therapeutic agent spends dissolved in the aqueous medium 240. Shortening the time period between dissolution and diffusion out of the device increases the likelihood that the therapeutic agent will remain stable because the time the agent spends in the aqueous media is minimized. This is important because many therapeutic agents have a finite, limited stability when dissolved in an aqueous media.

The same types of therapeutic agents 220 (e.g., interferon-alpha 2b for the treatment of Hepatitis C) and the same types of water-immiscible solvents 221 (e.g., perfluorodecalin) that were used with the first exemplary embodiment are compatible with this embodiment of the invention. Furthermore, the same type of microporous, membrane material (e.g., Whatman 1PS) can also be used to form the hydration membrane 250, except instead of a planar membrane (see FIG. 1), this embodiment utilizes a cylindrical, packet-shaped design to contain the particulate composition and the water-immiscible solvent.

A primary advantage (see FIG. 2) to the second embodiment is that the particles 220 are designed to float in the water-immiscible solvent 221 by sizing them to have a diameter>1 micrometer (i.e., non-colloidal), and by choosing a solvent density that is greater than the particle density. For example, the density of perfluorodecalin is approximately equal to 2.0, while the density of agent particles is normally between 1.1 and 1.5. When floating particles are placed in the packet, the particles are in substantially continuous contact with the hydrating membrane regardless of orientation of the implant housing 200. This characteristic is important in the use of the implant because the recipient of the device will likely be reclined for part of the day and upright for part of the day. Thus, unlike the implant device of FIG. 1, the implant device of FIG. 2 does not utilize a colloidal suspension, but rather utilizes the “particle in a packet” concept to address minimize the impact of gravitation forces on the operation of the device.

The housing 200 and the nanopore membrane 230 are manufactured in the same manner described for the first exemplary embodiment. Hydration membrane 250 is formed as a cylindrical with closed ends, or is formed into other capsule or packet shapes using means known in the art. In this embodiment, the packet-shaped microporous membrane 250 is filled with the water-immiscible liquid, rather than filling the interior chamber 210, as was the case with the exemplary embodiment of FIG. 1.

In alternate embodiments, a sintered plastic membrane is used to separate the phases within the device and act as the hydration membrane. Such membranes can (i) include different pore sizes, (ii) be molded/milled into useful shapes, and (iii) be either hydrophobic or hydrophilic in nature. Likewise, in alternate embodiments, the nanopore membrane may be either a microfabricated silicon nanopore membrane or a track-etch nanopore membrane.

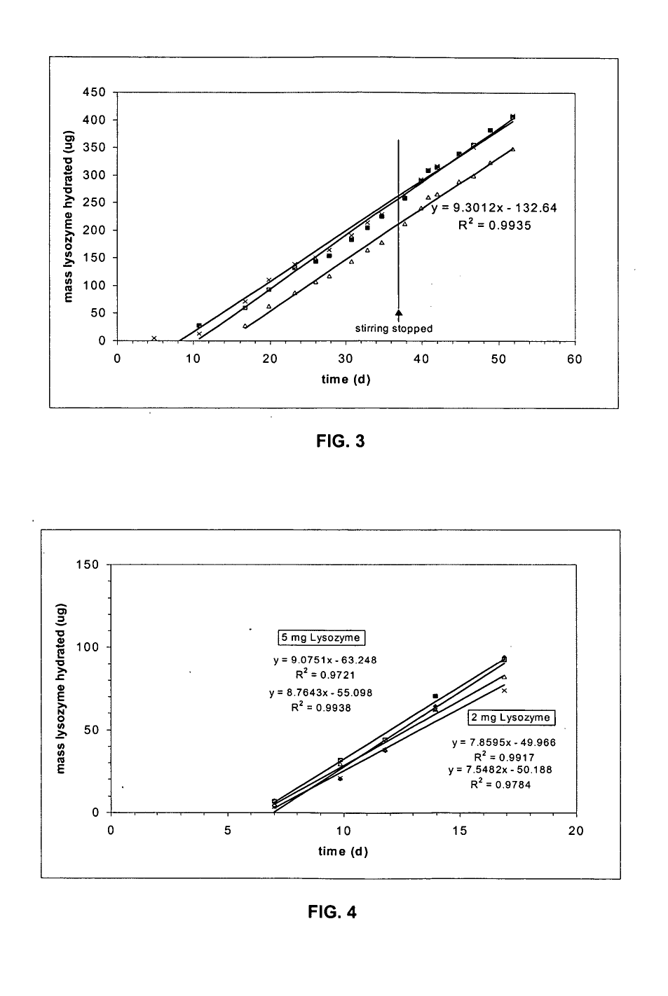

The data presented in FIG. 3 is illustrative of zero-order hydration of the surrogate molecule lysozyme using the planar version of the microporous membrane shown in FIG. 1. In this experiment, three identical acrylic chambers were used, each chamber including two subchambers separated by a planar microporous membrane. The microporous membrane was a Whatman 1PS Phase Separator having a diameter of 6 millimeters (area=113 mm²).

The lower portion of the chamber (i.e., the lower sub-chamber) was filled with 2.7 milliliters of the water-immiscible solvent, perfluorodecalin. Suspended in this solvent were 20 mg of solid lysozyme particles having an average particle size of 90 micrometers. The range of particle sizes was 75 to 105 micrometers. The suspended particles were observed to float in the perfluorodecalin and continuously contact the horizontal membrane surface. The upper portion of the chamber (i.e., the upper sub-chamber) contained 0.3 milliliters of phosphate buffered saline solution. A stirring bar was placed in the lower sub-chamber of each device to agitate the solvent at ambient temperature during the fifty-two day test period. After thirty-seven days, stirring was stopped to determine whether or not agitation had any effect on the hydration rate. The buffer solution in the upper sub-chamber of each device was removed each day and replaced with a fresh supply of solution. The lysozyme content of the upper sub-chamber samples was analyzed periodically to determine the quantity of lysozyme material that had dissolved in a one-day time period. The daily mass of lysozyme that was present in the upper sub-chamber was added to the sum of the previous days, thus the data in FIG. 3 represents the total mass of hydrated lysozyme.

As shown in FIG. 3, when loaded with 20 mg of solid lysozyme particles, the planar embodiment of the microporous membrane (see FIG. 1) hydrated the particles at an average rate of 9.3 micrograms per day (0.082 ug/day/mm²), after an initial delay of eight to fourteen days. Over the fifty-two day testing period shown in FIG. 3, however, only 2% of the lysozyme mass hydrated. Further, based on the data shown in FIG. 3, agitation, or the lack thereof appears to have an effect on the rate of release from the experimental device.

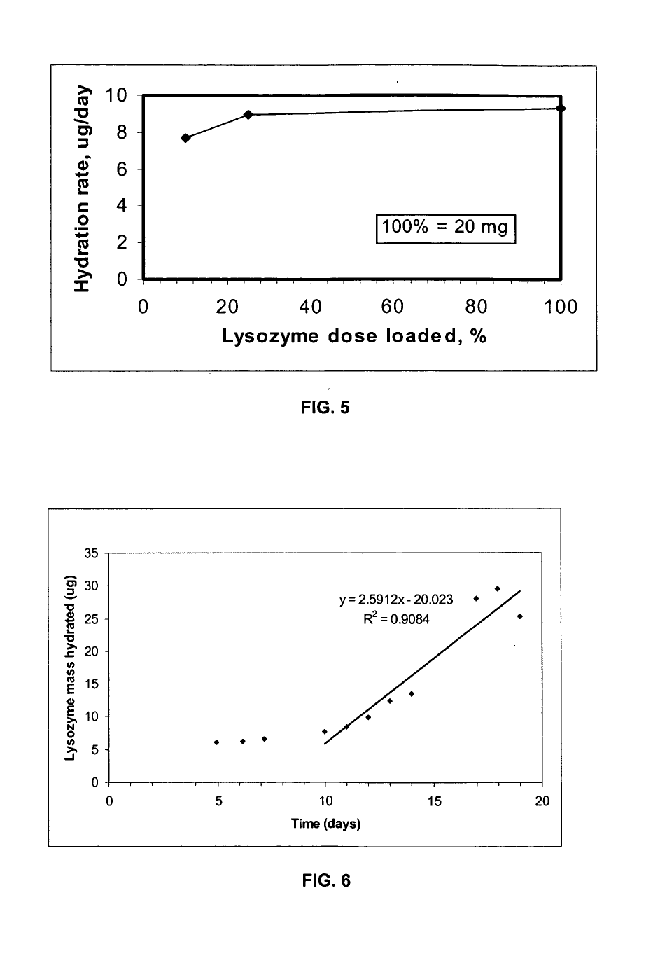

The effect of longer release periods is substantially equivalent to determining the release rate with decreasing amounts of lysozyme present in the device enclosure. To determine release variations over longer periods of time, additional planar-membrane, dual-chamber experiments were conducted using different initial amounts of lysozyme loaded, i.e., suspended, in the perfluorodecalin used to fill the lower sub-chamber. The results of the hydration analysis for 5 and 2 mg of suspended lysozyme are shown in FIG. 4. As indicated in FIG. 4, the hydration rate was 8.9 micrograms/day for 5 mg and 7.7 micrograms/day for 2 mg of suspended lysozyme. These average hydration rates are plotted in FIG. 5 as a function of the amount of mass loaded (100%=20 mg).

The data shown in FIG. 5 is predictive of what will occur at certain time points later in the release period when the device is initially loaded with 20 mg of lysozyme. These data indicate that the hydration rate will decrease by only 4% during the time the mass of lysozyme decreases by 80%. Even for only 2 mg loaded (10% of 20 mg), the rate only decreased to 83% of its original value. These results indicate that the linear release profiles shown in FIG. 3 are likely to continue even if only 10 to 20% of the lysozyme remains in the lower sub-chamber.

FIG. 6 provides hydration results for a device utilizing the three-dimensional capsule-like embodiment of the microporous membrane (see FIG. 2). For this experiment, a section of Whatman 1PS Phase Separator membrane was formed into a cylinder and sealed lengthwise and at both ends. The resulting cylinder was 10 mm in length and 4 mm in diameter (125 mm² membrane area). The cylinder was filled with approximately 1.4 mg of solid lysozyme having an average particle size of 90 micrometers combined with approximately 125 μl of the water-immiscible solvent perfluorodecalin,

The particle-filled cylinder described above was placed in a well containing 1.5 milliliters of phosphate buffered saline. A series of 70 μl samples were removed from the well at various time intervals. These samples were analyzed, and the results in FIG. 6 are the summation of mass hydrated as a function of time. Approximately 2.6 μg of lysozyme per day (i.e., 0.021 μg/mm²) was hydrated after a nine-day delay. This observed rate is a factor of four less than the hydration rate observed in the previously discussed planar membrane experiments; however, because the entire membrane area was not active for the cylinder-shaped membrane, the release per unit area data is in reasonable agreement bwtween the two experiments. Thus, the data shown in FIGS. 4-6 provide a clear indication that the devices shown in FIGS. 1 and 2 are capable of providing the long-term zero-order release desired for an implantable drug-delivery device.

Certain surfactants may be used to stabilize micronized dry powder or solid particulate suspensions, such as those utilized or compatible with the present invention. Surfactants suitable for this purpose include oleyl alcohol, oleic acid, synthetic dipalmitoylphosphatidylcholine, soybean lecithin, and sorbitan monooleate (Span 80).

Water soluble polymers are useful for improving the stability of certain peptide and protein therapeutics while in the aqueous phase of the present invention. Moreover, such polymers may be used to regulate the concentration of the protein therapeutic within the aqueous medium. Suitable polymers include polyethylene glycol of molecular weight 1000 to several million, such as, for example, PEG 2000 which is known to reduce the solubility of interferon alpha while not adversely affecting its stability or biological activity. Interferon may be precipitated with PEG 2000 and upon resolubilization, the interferon retains full biological activity. Polyvinylpyrrolidone and hyaluronic acid are also useful for this purpose.

Antioxidants may be added to the aqueous phase, i.e., aqueous medium, of the present invention to reduce the rate of oxidation of labile amino acid substituents of the therapeutic peptide/protein during its residence time in this phase. Suitable water soluble antioxidants are designed to be too large to diffuse through the nanopore membrane and include alpha tocopherol incorporated into an oil emulsion or liposome. Polymeric antioxidants are also useful for this purpose. Antioxidants may also be added to the perfluorocarbon, i.e., water immiscible solvent phase.

Certain excipients or materials exhibiting excipient properties may be added to the water-immiscible liquid used with the device of the present invention. For example, the inclusion of a an excipient with low water solubility that also exhibits INF-alpha stabilization properties can be used to limit water transfer, i.e., “sipping” or “imbibement,” through the microporous membrane, thereby enhancing the overall performance of the device.

1. An implantable device for sustained delivery of a therapeutic agent, comprising:
(a) a housing, wherein the housing further comprises an interior chamber and at least one aperture passing through the housing;
(b) a nanopore membrane in communication with the housing and covering the aperture, wherein the nanopore membrane further comprises a plurality of nanopore channels formed therein and passing though the membrane;
(c) a microporous membrane disposed within the housing beneath the nanopore membrane, the two membranes defining an interface therebetween;
(d) a first solvent disposed within the interface and in communication with both the nanopore membrane and the microporous membrane; and
(e) a particulate composition contained within the interior chamber below the microporous membrane, wherein the particulates are suspended in a mobile state in a second solvent, and wherein the second solvent is in communication with the microporous membrane.

2. The implantable device of claim 1, wherein the microporous membrane limits interaction between the particulates suspended in the second solvent and the first solvent, and wherein the limited interaction between the suspended particulates and the first solvent causes a portion of the particulate composition to dissolve into the first solvent and exit the device through the nanopore membrane.

3. The implantable device of claim 2, wherein the dissolution of the particulate composition into the first solvent produces a fixed amount of the particulate composition within the interface.

4. The implantable device of claim 2, wherein the dissolved particulate composition exits the device by diffusion through the nanopore membrane at a substantially constant rate.

5. The implantable device of claim 1, wherein the suspended particulates form a colloid in the second solvent.

6. The implantable device of claim 1, wherein the suspended particulates are stable within the second solvent for extended periods of time at 37° C.

7. The implantable device of claim 1, wherein at least one cross-sectional dimension of the nanopore channels is about 4 to 50 nanometers.

8. The implantable device of claim 1, wherein the nanopore membrane is at least one of a microfabricated silicon nanopore membrane and a track-etch nanopore membrane.

9. The implantable device of claim 1, wherein the microporous membrane further comprises pores of a pre-selected size, and wherein the particulate size of the suspended particulate composition agent is smaller in diameter than the diameter of the pores.

10. The implantable device of claim 1, wherein the microporous membrane is at least one of a hydrophobic phase separation membrane, a hydrophilic phase separation membrane.

11. The implantable device of claim 10, wherein the hydrophobic phase separation membrane is a Whatman 1PS Phase Separator.

12. The implantable device of claim 1, wherein the microporous membrane further comprises a sintered porous polymer, and wherein the sintered porous polymer has been treated to render it hydrophobic.

13. The implantable device of claim 12, wherein the sintered porous polymer further comprises at least one of sintered polyethylene and sintered polypropylene.

14. The implantable device of claim 1, wherein the first solvent is an aqueous medium.

15. The implantable device of claim 14, wherein the aqueous medium further comprises at least one of an antioxidant that cannot pass through the nanopore membrane, and a water-soluble polymer.

16. The implantable device of claim 15, wherein the water soluble polymer is polyethylene glycol, polyvinyl pyrrolidone, a polyol, a hyaluronic acid, or PEG 2000.

17. The implantable device of claim 1, wherein the second solvent is a water-immiscible liquid.

18. The implantable device of claim 17, wherein the water-immiscible liquid is a perfluorocarbon, a halocarbon oil, a dielectric fluid, or a polyol.

19. The implantable device of claim 17, wherein the water-immiscible liguid is perfluorodecalin, perfluoroperhydrophenanthrene, perfluoroperhydrofluorene, perfluoromethyldecalin, perfluorooctyl bromide, perfluoro-1,3-dimethylcyclohexane, perfluorotripropylamine, perfluorodichoroctane, perfluoromethylcyclohexylpiperidin, polychlorotrifluoroethylene, or polyoxyalkylene polyol.

20. The implantable device of claim 17, wherein the water-immiscible liquid further comprises an excipient for limiting water transfer through the microporous membrane.

21. The implantable device of claim 1, wherein the particulate composition further comprises a therapeutic agent.

22. The implantable device of claim 1, wherein the particulate composition is at least one of a dehydrated therapeutic agent and an emulsified therapeutic agent.

23. The implantable device of claim 1, wherein the particulate composition is a therapeutic polypeptide that is stable for about 2 to 3 weeks in an aqueous medium at about 37° C.

24. The implantable device of claim 23, wherein the therapeutic polypeptide is at least one of an interferon in crystalline form and an interferon in amorphous form.

25. The implantable device of claim 23, wherein the therapeutic polypeptide is suspended in the water-immiscible solvent in crystalline form or amorphous form.

26. The implantable device of claim 21, wherein the therapeutic agent is stabilized by the addition of at least one of a surfactant soluble in the water-immiscible solvent and a dehydrating agent.

27. The implantable device of claim 26, wherein the surfactant is a phospholipid, a oleyl alcohol, oleic acid, synthetic dipalmitoylphosphatidylcholine, soyvean lecitin, or sorbitan monooleate (Span 80).

28. The implantable device of claim 21, wherein the therapeutic agent is stabilized by the addition of an antioxidant incorporated into at least one of an oil emulsion or a liposome.

29. The implantable device of claim 28, wherein the antioxidant is alpha tocopherol.

30. The implantable device of claim 1, further comprising at least one object contained within the interior chamber for agitating the dehydrated particles when the device is moved.

31. An implantable device for sustained delivery of a therapeutic agent, comprising:
(a) a housing, wherein the housing further comprises an interior chamber and at least one aperture passing through the housing;
(b) a nanopore membrane in communication with the housing and covering the aperture, wherein the nanopore membrane further comprises a plurality of nanopore channels formed therein and passing though the membrane;
(c) a microporous membrane disposed within the housing, wherein the microporous membrane further comprises a capsule, and wherein the two membranes define an interface therebetween;
(d) a first solvent disposed within the interface and in communication with both the nanopore membrane and the microporous membrane; and
(e) a particulate composition contained within the microporous membrane, wherein the particulates are buoyant within the second solvent, and wherein the second solvent is in communication with the microporous membrane.

32. The implantable device of claim 31, wherein the microporous membrane limits interaction between the particulates suspended in the second solvent and the first solvent, and wherein the limited interaction between the buoyant particulates and the first solvent causes a portion of the particulate composition to dissolve into the first solvent and exit the device through the nanopore membrane.

33. The implantable device of claim 31, wherein the dissolution of the particulate composition into the first solvent produces a fixed amount of the particulate composition within the interface.

32. The implantable device of claim 31, wherein the dissolved particulate composition exits the device by diffusion through the nanopore membrane at a substantially constant rate.

33. The implantable device of claim 31, wherein the suspended particulates are stable within the second solvent for extended periods of time at 37° C.

34. The implantable device of claim 31, wherein at least one cross-sectional dimension of the nanopore channels is about 4 to 50 nanometers.

34. The implantable device of claim 31, wherein the nanopore membrane is at least one of a microfabricated silicon nanopore membrane and a track-etch nanopore membrane.

35. The implantable device of claim 31, wherein the microporous membrane further comprises pores of a pre-selected size, and wherein the particulate size of the suspended particulate composition agent is smaller in diameter than the diameter of the pores.

36. The implantable device of claim 31, wherein the microporous membrane is at least one of a hydrophobic phase separation membrane, a hydrophilic phase separation membrane.

37. The implantable device of claim 36, wherein the hydrophobic phase separation membrane is a Whatman 1PS Phase Separator.

38. The implantable device of claim 31, wherein the microporous membrane further comprises a sintered porous polymer, and wherein the sintered porous polymer has been treated to render it hydrophobic.

39. The implantable device of claim 38 wherein the sintered porous polymer further comprises at least one of sintered polyethylene and sintered polypropylene.

40. The implantable device of claim 31, wherein the first solvent is an aqueous medium.

41. The implantable device of claim 40, wherein the aqueous medium further comprises at least one of an antioxidant that cannot pass through the nanopore membrane, and a water-soluble polymer.

42. The implantable device of claim 41, wherein the water soluble polymer is polyethylene glycol, polyvinyl pyrrolidone, a polyol, a hyaluronic acid, or PEG 2000.

43. The implantable device of claim 31, wherein the second solvent is a water-immiscible liquid.

44. The implantable device of claim 43 wherein the water-immiscible liquid is a perfluorocarbon, a halocarbon oil, a dielectric fluid, or a polyol.

45. The implantable device of claim 43, wherein the water-immiscible liguid is perfluorodecalin, perfluoroperhydrophenanthrene, perfluoroperhydrofluorene, perfluoromethyldecalin, perfluorooctyl bromide, perfluoro-1,3-dimethylcyclohexane, perfluorotripropylamine, perfluorodichoroctane, perfluoromethylcyclohexylpiperidin, polychlorotrifluoroethylene, or polyoxyalkylene polyol.

46. The implantable device of claim 43 wherein the water-immiscible liquid further comprises an excipient for limiting water transfer through the microporous membrane.

47. The implantable device of claim 31, wherein the particulate composition further comprises a therapeutic agent.

48. The implantable device of claim 31, wherein the particulate composition is at least one of a dehydrated therapeutic agent and an emulsified therapeutic agent.

49. The implantable device of claim 31, wherein the particulate composition is a therapeutic polypeptide that is stable for about 2 to 3 weeks in an aqueous medium at about 37° C.

50. The implantable device of claim 49, wherein the therapeutic polypeptide is at least one of an interferon in crystalline form and an interferon in amorphous form.

51. The implantable device of claim 49, wherein the therapeutic polypeptide is suspended in the water-immiscible solvent in crystalline form or amorphous form.

52. The implantable device of claim 47, wherein the therapeutic agent is stabilized by the addition of at least one of a surfactant soluble in the water-immiscible solvent and a dehydrating agent.

53. The implantable device of claim 52, wherein the surfactant is a phospholipid, a oleyl alcohol, oleic acid, synthetic dipalmitoylphosphatidylcholine, soyvean lecitin, or sorbitan monooleate (Span 80).

54. The implantable device of claim 47, wherein the therapeutic agent is stabilized by the addition of an antioxidant incorporated into at least one of an oil emulsion or a liposome.

55. The implantable device of claim 54, wherein the antioxidant is alpha tocopherol.

56. The implantable device of claim 31, further comprising at least one object contained within the interior chamber for agitating the dehydrated particles when the device is moved.

57. The implantable device of claim 31, wherein the microporous membrane comprises a cylinder capped at the ends and having pores of a pre-selected size, and wherein the size of the particulates causes the particulates to contact the microporous membrane due to buoyancy.
